(19) United States
(12) Patent Application Publication
(10) Pub. No.: US 2025/0294648 A1
(43) Pub. Date: Sep. 18, 2025

(54) APPARATUS FOR DISPENSING VOLATILE SUBSTANCES AND METHOD FOR PRODUCING THE SAME

(71) Applicant: CTR, LDA, Samora Correia (PT)

(72) Inventors: Teruo HISHIKI, Tokyo (JP); Harold AUGIER, Zhongshan City (CN)

(21) Appl. No.: 18/863,381

(22) PCT Filed: May 6, 2022
(86) PCT No.: PCT/EP2022/062364

§ 371 Date: Nov. 6, 2024

(51) Int. Cl. H05B 3/78

(52) U.S. Cl. CPC H05B 3/78

(57) ABSTRACT

A device and method for dispensing volatile substances include a container for the substance, at least one capillary element at least partially in the container, in contact with the substance and conveying it by capillary action to at least one substance dispensing area, an electrical heating device having at least one electrical heating resistor the region of the capillary element, and an electrical connection element supplying the electrical heating device with electrical energy. The heating resistor is formed by at least two element rows spaced apart from one another, each element row having at least one areal heating element, preferably at least one PTC areal heating element, the areal heating elements are applied as film layer resistors to an electrically non-conductive base carrier layer, and an electrically conductive film layer conductor grid is applied to the base carrier layer for supplying electrical energy to the areal heating elements.

The invention relates to a device for dispensing, in particular for evaporating, volatile substances, in particular fragrances and/or active substances, according to the preamble of claim 1. Furthermore, the invention relates to a method for manufacturing such a device, in particular for manufacturing a heating device of such a device, according to the preamble of claim 22.

A known device for dispensing, in particular for evaporating volatile substances, is known from WO 2017/2157287 A1 and from U.S. Pat. No. 5,903,710 A and has a container for the substance to be dispensed and a capillary element which is arranged at least partially in the container and which is in contact with the substance to be dispensed and conveys it by means of capillary action to at least one substance dispensing area. Furthermore, an electrical heating device is provided which has an electrical heating resistor in the region of the capillary element which is in contact with the substance dispensing area. In addition, a container holder is provided to which the container can be connected and which has an electrical connection element by means of which the electrical heating device can be supplied with electrical energy. The electrical connection element is specifically designed here as a plug component with projecting plug contacts that can be plugged into an electrical socket for electrical power supply.

In the aforementioned prior art, the electrical heating resistor is represented as a relatively large areal element, which can also be formed, among other things, by spraying or printing and drying an electrically conductive ink or paint that has a resistance function. As a disadvantage, uneven temperature distributions can occur here due to the large areal area, in particular with temperature hot spots, which can lead to damage in the capillary element, especially at initial, high switch-on temperatures. In particular, a free capillary element end can dry out and dehumidify considerably as a result of temperature hot spots, whereby the capillary structure can be damaged by sticking and/or caking together to such an extent that the delivery rate for the substance to be dispensed is considerably reduced and, in extreme cases, no more substance evaporates and a partially still filled container has to be replaced. Moreover, the production of a relatively large heating element area using an electrically conductive ink or paint is difficult in terms of production with regard to uniform distribution and drying and can lead to uneven layer thicknesses, which in turn promote uneven temperature distribution in conjunction with temperature hot spots. In addition, the prior art does not provide any specific information on how to implement the simplest possible and most functionally reliable contacting for supplying energy to the electrical, planar heating resistor there.

It is the object of the invention to create a device for dispensing, in particular for evaporating, volatile substances, in particular fragrances and/or active ingredients, with regard to its electrical heating device in such a way that gentle and long-lasting operation of the capillary element with a high degree of evaporation is possible with a simple and inexpensive structure as well as simple manufacture. A further object of the invention is to propose a manufacturing method suitable for this purpose.

The object of the invention is solved with respect to the device with the features of independent claim 1. Advantageous embodiments thereof are the subject of the subclaims referring back thereto.

According to claim 1, there is provided a device for dispensing volatile substances comprising:
a container for the substance to be dispensed,
at least one capillary element which is arranged at least partially in the container and which is in contact with the substance to be dispensed and conveys it by means of capillary action to at least one substance dispensing area,
an electrical heating device with at least one electrical heating resistor arranged in the region of the capillary element,
an electrical connection element by means of which the electrical heating device can be supplied with electrical energy.

According to the invention, it is further provided:
that the heating resistor is formed by at least two element rows spaced apart from one another, each element row having at least one areal heating element, preferably at least one PTC areal heating element,
that the areal heating elements are applied as film layer resistors to an electrically non-conductive base carrier layer,
that an electrically conductive film layer conductor grid is applied to the base carrier layer for supplying electrical energy to the areal heating elements.

It has been shown that with the claimed division of the electrical heating resistor into spaced element rows and individual areal heating elements, a uniform temperature distribution in the area of the capillary element can be achieved, in particular without harmful temperature hot spots. This avoids any harmful influence on the capillary element and can ensure uniform, safe operation of the device.

The areal heating elements are preferably PTC areal heating elements, i.e. heating elements whose material has a positive temperature coefficient, which means that their electrical resistance also increases with rising temperature. Specifically, this means that after the device is switched on, the PTC function of the PTC areal heating elements does not yet take effect immediately, so that a comparatively high temperature of, for example, about 70° C. to 110° C. is initially generated with the heating device, depending on the choice of materials used and the design structure. For this purpose, an electrical power of 5 watts may be required as an example. As the temperature rises, this is reduced again by the use of the PTC function, for example to operating temperatures of approx. 60° C., whereby the required electrical power then also decreases, for example to 1 watt.

Further, according to a particularly preferred specific embodiment of the device, it is provided:
that a first main line and a second main line of the film layer conductor grid are arranged on opposite sides of the at least two rows of elements and can each be connected to poles of different voltage of the electrical connection element,
that, starting from the first main line, a respective stub line runs along each row of elements and makes electrical contact with the at least one areal heating element of each row of elements,
that, starting from the second, opposite main line, a respective stub line runs along each row of elements and makes electrical contact with the at least one areal heating element of each row of elements, and
in that the stub lines of the first main line and the stub lines of the second main line each have a stub line distance between assigned areal heating elements of an element row.

In addition to being easy to manufacture, such a design also results in a particularly advantageous compact arrangement with functionally reliable contacting and power supply.

The power supply, and in particular a DC power supply, depends on the material used for the areal heating elements and can be, for example, the residential voltage or a reduced voltage, e.g. in the range of 5 to 12V, for e.g. PTC ink materials.

Moreover, the above advantages can be achieved in particular if, according to a particularly preferred embodiment, at least two areal heating elements spaced apart from each other with intermediate distances are arranged in an element row and/or an equal number of areal heating elements is provided in each element row.

The production, in particular by a printing or spraying process, with electrically conductive ink or electrically conductive paint can be carried out more easily, more reliably and more uniformly with an arrangement of relatively small areal heating elements compared with large-area applications. A particular advantage also lies in the provision of an electrically conductive film layer conductor grid, which can also be produced simply and inexpensively and results in a functionally reliable electrical bond between the electrical conductors and the areal heating elements. Electrical contacting is thus achieved here by means of a relatively large-area contacting composite, i.e. by means of intensive contact without weak contact points and tolerances.

In principle, there are various options as to how or in which order the areal heating elements and the film layer conductor grid can be applied to the base carrier layer: According to a first variant, the areal heating elements can be applied to the base carrier layer before the film layer conductor grid and thus lie below the stub lines subsequently applied to the base carrier layer. Alternatively, the film layer conductor grid can be applied to the base carrier layer before the areal heating elements, and then the areal heating elements can be applied to the base carrier layer so that the areal heating elements lie on top of the stub lines.

According to a particularly preferred embodiment, the areal heating elements are formed by an electrically conductive ink or paint having a resistance function, preferably a PTC resistance function, which is applied to the base carrier layer as a film layer, preferably as a thin film layer, preferably printed and dried.

Furthermore, the electrically conductive film layer conductor grid can also be formed by an electrically conductive ink or ink having a current-carrying function, preferably having silver, which is applied to the base carrier layer as a film layer, preferably as a thin film layer, preferably printed and dried.

In a particularly preferred embodiment, the areal heating elements each have the same design and/or the areal heating elements have a rectangular shape, in particular a square shape, whereby it is preferably provided that the areal heating elements have edge lengths of 2 mm to 8 mm. Rectangular areal heating elements can be easily calculated and designed and therefore ensure high quality and precision. The specified edge lengths have also proved to be particularly advantageous limits in conjunction with specific arrangements, with which, on the one hand, a desired heating output can be achieved and, on the other hand, undesirable hot spots can be reliably avoided.

According to a further particularly preferred concrete embodiment, two to six, preferably two to four, element rows, each with two to five, preferably three or four, areal heating elements, are arranged in parallel, preferably with a respective equal row spacing between mutually opposite areal heating elements of adjacent element rows and/or with a respective equal intermediate spacing between adjacent areal heating elements of an element row. This makes it possible to generate uniform areal patterns which, on the one hand, can be produced simply and inexpensively and, on the other hand, ensure a favourable, uniform temperature distribution in the region of the at least one capillary element.

In order to achieve particularly suitable results with a very compact structure, the row spacing defined by the distance between two adjacent areal heating elements of adjacent element rows is preferably 1 mm to 5 mm. Alternatively or additionally, the distance between two adjacent areal heating elements of an element row is 0.5 mm to 5 mm.

For the required, functionally reliable contacting with a compact structure, the first main line and the second main line of the film layer conductor grid are preferably each aligned laterally and essentially perpendicular to the element rows and have a main line width of 1 mm to 5 mm, preferably of 1 mm to 3 mm. Alternatively or additionally, for the same reason, the stub lines can be aligned parallel and associated with the element rows, and preferably with a comparatively smaller stub line width of 0.3 mm to 1 mm.

Particularly preferably, the two stub lines of an element row have a stub line spacing of at least 1 mm or 1 mm or greater, with the stub line spacing between the two stub lines always being selected so that the two stub lines run within the area of the associated areal heating elements. This means that in each case areas of the areal heating elements project beyond the stub lines, so that the stub lines have a secure contact bond with the associated areal heating elements over their entire width even in the event of tolerances in manufacture.

A particularly compact and material-saving design of the heating device, while still providing the desired heating power in a functionally reliable manner, can be achieved if the layer thickness of the areal heating elements formed by film layer resistors and/or of the film layer conductor grid is 0.5 μm to 30 μm, preferably 5 μm to 25 μm, most preferably 10 μm to 20 μm.

With the dimensions and arrangements explained above, the evenly distributed temperatures suitable for operation of the device can be realized particularly well, especially when using commercially available inks or paints for the production of the areal heating elements and the film layer conductor grid.

For example, materials that available on the market as inks can be used for the production of both the areal heating elements and the film layer conductor grid, which are specifically applied to the base carrier layer with a layer or film thickness of approx. 15 μm. For example, Loctite® ECI 8001 or Loctite® 8120 can be used to produce the areal heating elements and Loctite® ECI 1010 or Loctite® ECI 4002 can be used to produce the film layer conductor grid. More generally, the base material for the areal heating elements is preferably a PET material or a polyamide material, the latter being more suitable for higher temperatures.

In the following, two particularly preferred but different and alternative devices are explained by way of example only:

In the first embodiment, the base carrier layer is formed by the container or is a component of the container. Further, a container holder is provided to which the receptacle is detachably connectable, the container holder further comprising the electrical connection element to which the receptacle is electrically connectable. Such a structure is simple to manufacture and takes up relatively little space. To establish the electrical connection with the areal heating elements, an electrical contact device, preferably a contact device with plug segments, is provided on the main lines, by means of which the two main lines for supplying electrical power to the areal heating elements can be connected directly or indirectly to the electrical connection element.

Further, in this first embodiment, it is provided that the container holder is configured as a plug component, wherein the electrical connection element has protruding plug contacts that can be plugged into an electrical socket for supplying electrical power to the areal heating elements and for holding the plug component as well as the container connected to the plug component.

In this first embodiment, the base carrier layer can be directly a non-conductive outer side of a container wall part or a non-conductive intermediate layer connected to a container wall part, with plastic material preferably being used here. In a heat transfer region, a capillary element region to be heated as a substance dispensing area is to be adjacent to the base carrier layer with the areal heating elements, if necessary with the interposition of an areal heat transfer element, in particular in areal contact.

Particularly suitable for this purpose is a capillary element which is designed as a U-shaped, flat wick which is in contact with the substance to be dispensed by means of both U-legs in the container, the U-base part forming the substance dispensing area and this resting against a heat transfer area. For the release of the substance to the environment, a substance outlet opening which can be opened for the operating state is also preferably arranged in the otherwise closed container.

In an alternative, second embodiment, a container holder is provided to which the container can be detachably connected, the container holder being formed by a housing component in which the container is at least partially accommodated. In addition, the base carrier layer is here, together with the areal heating elements, a component of the housing component and is arranged in the interior of the housing component in the region of the inserted container, the housing component having the electrical connection element to which the container can be electrically connected.

In this second embodiment, it is preferably provided that the base carrier layer with the areal heating elements is arranged in the region of a substance dispensing area of a capillary element to be heated, most preferably associated with a free wick end of a wick projecting from the container as a capillary element.

Furthermore, in this second embodiment, it is preferably provided that the housing component is designed as a plug component, the electrical connection element having projecting plug contacts which can be plugged into an electrical socket for supplying electrical power to the areal heating elements and for holding the plug component and the container connected to the plug component. In this embodiment, the base carrier layer with the areal heating elements is thus an integral part of the housing component and not of the container. The areal heating elements can also be attached, for example, to a ring heating element as the base carrier layer, which surrounds a free wick end projecting from the container.

The object of the invention is solved with respect to the method with the features of independent claim 22. Advantageous embodiments thereof are the subject of the subclaims referring back thereto.

The advantages resulting here are identical to those of the device, so that to avoid repetition, reference is made to the explanations previously given concerning the device.

The invention is further explained by way of example with the help of the following figures:

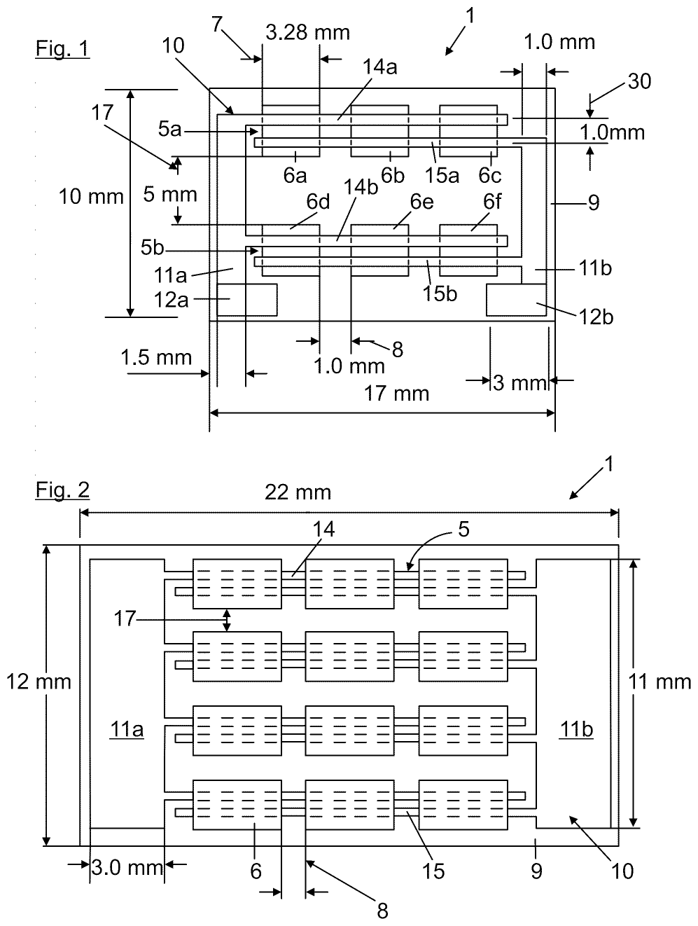

FIG. 1 an electrical, areal heating resistor with PTC areal heating elements of a first embodiment;

FIG. 2 an electrical, areal heating resistor with PTC areal heating elements of a second embodiment;

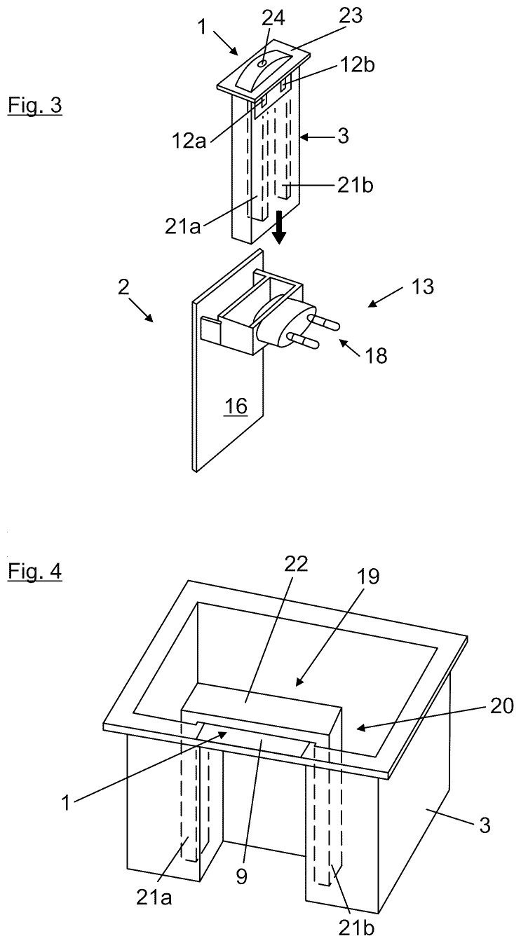

FIG. 3 a schematic illustration of a container holder and a container;

FIG. 4 a perspective view of a modified container obliquely from above with a capillary element and a heating resistor;

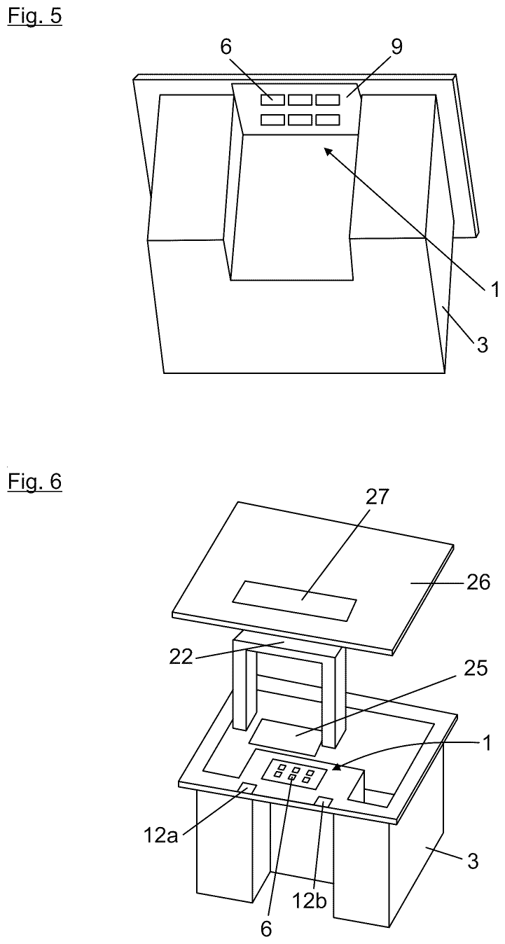

FIG. 5 a representation of the container according to FIG. 4, viewed obliquely from below;

FIG. 6 an exploded view of the container according to FIG. 4;

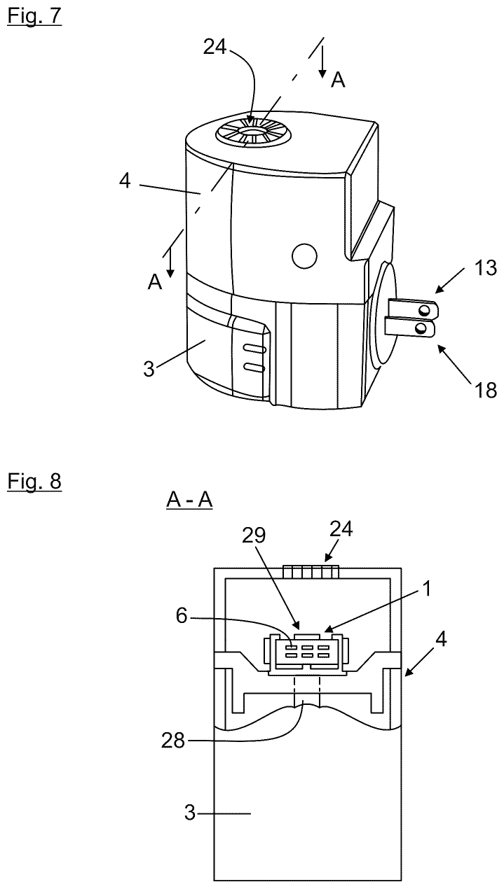

FIG. 7 an alternative embodiment to the illustrations according to FIGS. 3 to 6 with a container holder as a housing component;

FIG. 8 a partial section along line A-A of FIG. 7.

FIGS. 1 and 2 show a plan view of two different embodiments of an exemplary electrical and areal heating resistor of an electrical heating device, which are used in a device for dispensing volatile substances, in particular fragrances and/or active ingredients, which has a container 3 for the substance to be dispensed.

In a first embodiment of such a device, also referred to as a dispenser, shown in FIGS. 3 to 6 in this respect, a container holder is designed as a plug component 2, the heating resistor 1 being a component of the container 3 here by way of example, or in other words being integrated into the container 3.

In an alternative embodiment of such a dispensing device, as shown in FIGS. 7 and 8, the container holder is formed as a housing component 4 into which the container 3 is inserted, the heating resistor 1 not being a component of the container 3 here, but being a component of the housing component 4.

Referring to FIG. 1, the areal heating resistor 1 has a plurality of element rows 5, here exemplarily two, spaced apart from each other, hereinafter referred to as element rows 5a and 5b. These element rows 5a, 5b each have, here exemplarily, a plurality of, for example three, spaced-apart areal heating elements 6, which are hereinafter referred to as areal heating elements 6a, 6b, 6c and 6d, 6e, 6f and are which are here further exemplarily PTC areal heating elements. These PTC areal heating elements 6a to 6f preferably each have the same geometry or shape, here exemplarily each having the same rectangular or square shape. The square-shaped PTC areal heating elements 6a to 6f can, for example, have an edge length 7 of 3.28 mm and, for example, the intermediate distance 8 between the individual PTC areal heating elements 6a to 6f within an element row 5a, 5b is approximately 1 mm. The row spacing 17 between the oppositely assigned PTC areal heating elements 6a to 6f of the element rows 5a, 5b is approximately 5 mm.

It has been shown that, in general, distances (intermediate distances, row distances) greater than 5 mm increase the risk of damaging temperature hot spots and, accordingly, smaller distances (intermediate distances, row distances) of approximately 1 mm are particularly suitable for forming the heating resistor 1.

The PTC areal heating elements 6a to 6f are preferably formed by film layer resistors, which are preferably produced by an electrically conductive ink or paint having a resistance function, preferably a PTC resistance function, and are printed and dried as a film layer on a base carrier layer 9. For example, such an electrically conductive ink or paint is available on the market under the designation Loctite® ECI 8001 or Loctite® ECI 8120, which has the advantage that it can be applied to the base carrier layer 9 with a very thin film layer thickness, e.g. with a film layer thickness of 15 μm. The PTC function advantageously leads here with a relatively high switch-on temperature of, for example, about 70° C. to 110° C. to a rapid substance release after switching on, the temperature then subsequently decreasing to a desired operating temperature, for example to about 60° C., due to the effectiveness of the PTC function and an associated reduction in the electrical power consumption, for example from about 5 watts to about 1 watt of operating power.

For the electrical power supply of the PTC areal heating elements 6a to 6f, an electrically conductive film layer conductor grid 10 is applied to the base carrier layer 9, e.g. also with a film layer thickness of 15 μm. Similar to the PTC areal heating elements 6a to 6f, this film layer conductor grid 10 is printed onto the base carrier layer 9 by means of an ink or paint and dried, whereby here the ink or paint has an electrically conductive function, which is preferably due to silver or copper particles. For example, such materials are available on the market under the designation Loctite® ECI 1010 or Loctite® ECI 4002.

The film layer conductor grid 10 has a first main line 11a and a second main line 11b which, in the example shown here, each run opposite one another laterally and transversely to the two rows of elements 5a, 5b and which can each be connected to poles of different voltage of an electrical connection element, e.g. can be connected to poles of different voltage of an electrical connection element of a container holder, as will be explained in more detail below with reference to the individual embodiment examples:

For example, if the heating resistor 1 is arranged on the container 3 in an embodiment according to FIGS. 3 to 6, an electrical contact device with plug elements 12a, 12b can also be provided on the first main line 11a and on the second main line 11b, respectively, with which an electrical contact can be made to the electrical connection element 13 thereof when the container 3 is connected to the plug component 2 acting as a container holder.

Here, for example, the first main line 11a and the second main line 11b have a width of approx. 1 mm. Starting from the first main line 11a, two stub lines 14a, 14b run in the example shown here with an electrical contact and interconnection via the PTC areal heating elements 6a, 6b, 6c and via the PTC areal heating elements 6d, 6e, 6f, respectively.

Starting from the second main line 11b, two stub lines 15a, 15b also run with an electrical contact and interconnection via the PTC areal heating elements 6a, 6b, 6c and via the PTC areal heating elements 6d, 6e, 6f of each element row 5a and 5b, respectively. The stub lines 14a, 15a and the stub lines 14b, 15b of each element row 5a and 5b, for example, each have a stub line spacing 30 of approx. 1 mm and thus run well within the area of the PTC areal heating elements 6a to 6f, so that a reliable contact overlap is ensured even with manufacturing tolerances. The stub lines 14a, 14b and 15a, 15b have a smaller stub line width than the main lines 11a, 11b, for example approx. 0.3 mm.

The second embodiment shown in FIG. 2 of a flat heating resistor 1 which can be used in a dispensing device is constructed similarly to the heating resistor 1 in FIG. 1, so that only the differences will be explained below, using largely the same reference signs:

A significant difference here is that the film layer conductor grid 10 was printed onto the base carrier layer 9 before the PTC areal heating elements 6, and then the PTC areal heating elements 6 were produced in a further printing process, as a result of which the film layer conductor grid 10 is located below the PTC areal heating elements 6, with a very good and reliable electrical contact bond, as in FIG. 1.

Instead of the merely exemplary two element rows 5a, 5b according to FIG. 1, four parallel element rows 5 are provided in the embodiment according to FIG. 2—again merely exemplary—which here likewise merely exemplarily each have three PTC areal heating elements 6. In contrast to FIG. 1, the row spacing 17 between the two element rows 5a, 5b in the embodiment according to FIG. 2 is somewhat smaller here merely to illustrate the basic possibility of varying the spacing, but could also be the same or somewhat larger. The PTC areal heating elements 6 each have a rectangular shape here merely by way of example, with the element length, viewed in the direction of the element rows, being, for example, approximately 3 mm and/or with the element width, viewed transversely to the element row, being, for example, approximately 2 mm.

With both the embodiment of the heating resistor 1 according to FIG. 1 and FIG. 2, the required small dimensions of the entire heating resistor arrangement of about 10 to 12 mm with suitable heating power for accommodation in a dispenser can be realized.

FIG. 3 schematically shows the first embodiment of the device as a dispenser for volatile substances, in particular fragrances and/or active ingredients. In this embodiment, the container holder for the container 3 is designed as a plug component 2, in which an electrical connection element 13 is mounted on a cover plate 16. The cover plate 16 may have design elements (not shown) in the visible area, wherein the electrical connection element 13 also has plug contacts 18 which can be plugged into an electrical socket (not shown) for supplying electrical power to the PTC areal heating elements 6 and for holding the plug component 2 with the container 3. The substance to be dispensed, in particular in the form of a liquid, is accommodated in the container 3, with a capillary element 19, which is designed here merely by way of example as a U-shaped flat wick 20 with U-legs 21a, 21b, being immersed so that its upper U-base region forms a substance dispensing region 22 which is to be heated by means of the heating resistor 1.

In FIG. 3, the substance dispensing area 22 and also the heating resistor 1 are exemplarily covered with a cover hood 23, which has an outlet opening 24 for the substance to be dispensed.

FIG. 4 shows a container 3 with a slightly modified design compared to FIG. 3 in the open top state, in which the substance to be dispensed is accommodated during operation. Here, too, a U-shaped flat wick 20 is shown as capillary element 19, which is immersed with its U-legs 21a, 21b downward into the substance to be dispensed. The U-base as substance dispensing area 22 rests here from above on an electrically non-conductive base carrier layer 9 of an upper shoulder or projection of the container 3. In the embodiment shown in FIG. 4, the flat heating resistor 1 with the PTC areal heating elements 6 is arranged on the base carrier layer 9 under the substance delivery area 22 (not visible), whereby the substance delivery area 22 here rests directly on and against the electrical heating resistor 1.

FIG. 5 shows a modified embodiment of the container 3 shown in FIG. 4 with regard to the arrangement of the heating resistor 1. Here, the base carrier layer 9 with the PTC areal heating elements 6 is attached under the upper wall shoulder on which the substance dispensing area 22 of the flat wick 20 rests. This means that heat is transferred here by conduction through the upper wall shoulder to the substance dispensing area 22. The base carrier layer 9 can therefore also be an electrically non-conductive wall area of the container 3 on which the heating resistor 1 is printed.

FIG. 6 again shows schematically in an exploded view a modified embodiment of the container 3. Here, too, the heating resistor 1 with the PTC areal heating elements 6 is arranged on an upper wall shoulder of the container 3 and is equipped with plug-in elements 12a, 12b for connection to electrical connection elements 17 of a plug component 2 acting as a container holder. A heat-conducting, preferably thin plate 25, for example an aluminium plate, is arranged here on the heating resistor 1 and under the substance dispensing area 22 for uniform heat distribution to the substance dispensing area 22. The container 3 is here only exemplarily covered from above with a tightly attached, flat cover element 26, which has a dispensing opening 27 above the substance dispensing area 22. The dispensing opening 27, or as shown in FIG. 3 as a smaller outlet opening 24, is still closed in the sales packaging of the container and can be opened for operation before the container 3 is inserted into the plug component 2, in particular by tearing open a film seal.

In an alternative embodiment of the dispenser device shown in FIG. 7, the container holder is designed as a housing component 4 and, in addition, here as a plug component by way of example, which has an electrical connection element 13 with plug contacts 18 which can be plugged into an electrical socket (not shown) for an electrical power supply to the PTC areal heating elements 6 and for holding the housing component 4 and the container 3 which is also partially inserted therein.

From the sectional view according to FIG. 8, it is also evident that the container 3 is partially inserted into the housing component 4 from below and connected to it in a manner known per se, with a wick 28 in the form of a capillary element projecting upwards from the container 3 with its free wick end 29. In the region of the free wick end 29, the heating resistor 1 with the PTC areal heating elements 6 is fixedly arranged here in a schematic representation as a component of the housing component 4. Depending on the local conditions, the base carrier layer 9 with the PTC heating elements 6 can be arranged in the area of the free wick end 29 next to it or, if necessary, at least partially surrounding it. In particular, the heating resistor with the PTC areal heating elements 6 can be arranged on a ring part, for example a ceramic ring part known per se, through which the free wick end 29 is inserted during operation. In the upper region of the housing component 4 above the free wick end 29, an outlet opening 24 is arranged for the substance to be discharged into the environment.

### LIST OF REFERENCE SIGNS

1 Electrical, flat heating resistor
2 Plug component
3 Container
4 Housing component
5a, b Element rows
6a, b, c, d, e, f, PTC areal heating elements
7 Edge length
8 Intermediate distance
9 Base carrier layer
10 Film layer conductor grid
11a, b First and second main line
12a, b Plug-in elements
13 Electrical connection element
14a, b Stub lines from first main line
15a, b Stub lines from second main line
16 Cover plate
17 Row spacing
18 Plug-in contacts
19 Capillary element
20 Flat wick
21a, b U-leg
22 Substance dispensing area
23 Cover
24 Outlet opening
25 Heat conductive plate
26 Cover element
27 Delivery opening
28 Wick
29 Free wick end
30 Stub line distance

1-29. (canceled)

30. A device for dispensing volatile substances, the device comprising:
a container for the substance to be dispensed;
at least one substance dispensing area;
at least one capillary element disposed at least partially in said container, being in contact with the substance to be dispensed and conveying the substance to be dispensed by capillary action to said at least one substance dispensing area;
an electrical heating device having at least one electrical heating resistor disposed in a region of said at least one capillary element;
an electrical connection element for supplying said electrical heating device with electrical energy;
said at least one electrical heating resistor being formed by at least two element rows spaced apart from one another, each of said at least two element rows having at least one areal heating element;
an electrically non-conductive base carrier layer, said areal heating elements being applied as film layer resistors to said electrically non-conductive base carrier layer; and
an electrically conductive film layer conductor grid applied to said electrically non-conductive base carrier layer for supplying electrical energy to said areal heating elements.

31. The device according to claim 30, wherein said areal heating elements are PTC areal heating elements.

32. The device according to claim 30, wherein:
said electrical connection element has poles of different voltage;
said film layer conductor grid has a first main line and a second main line disposed on opposite sides of said at least two rows of elements, said first and second main lines configured to be connected to said poles of different voltage of said electrical connection element;
stub lines each starting from said first main line, running along a respective one of said rows of elements and making electrical contact with said at least one areal heating element of each of said rows of elements;
other stub lines each starting from said second, opposite main line, running along a respective one of said rows of elements and making electrical contact with said at least one areal heating element of each of said rows of elements; and
said stub lines starting from said first main line and said other stub lines starting from said second main line each have a stub line distance between associated areal heating elements of an element row.

33. The device according to claim 30, wherein said at least one areal heating element includes at least one of:
at least two areal heating elements disposed in one of said element rows and spaced apart from one another by intermediate distances, or
an equal number of areal heating elements provided in each of said element rows.

34. The device according to claim 32, wherein said stub lines starting from said first main line and said other stub lines starting from said second main line each run with an electrical contact through an interconnection below or above said areal heating elements of each of said rows of elements.

35. The device according to claim 30, wherein said areal heating elements applied as film layer resistors are formed of an electrically conductive ink or paint having a resistance function or a PTC resistance function, and are applied to said base carrier layer as a film layer or are printed and dried.

36. The device according to claim 30, wherein said electrically conductive film layer conductor grid is formed of an electrically conductive ink or paint having a current-carrying function and being applied to said base carrier layer as a film layer or printed and dried.

37. The device according to claim 36, wherein said electrically conductive ink or paint of said electrically conductive film layer conductor grid includes silver.

38. The device according to claim 30, wherein said areal heating elements each have at least one of an identical shape, or a rectangular shape, or a square shape, or edge lengths of 2 mm to 8 mm.

39. The device according to claim 30, wherein two to six of said element rows each having two to five of said areal heating elements:
are disposed in parallel, or
each have the same row spacing between mutually opposite areal heating elements of adjacent element rows, or
each have the same intermediate spacing between adjacent areal heating elements of an element row.

40. The device according to claim 30, wherein two to four of said element rows each having three or four of said areal heating elements:
are disposed in parallel, or
each have the same row spacing between mutually opposite areal heating elements of adjacent element rows, or
each have the same intermediate spacing between adjacent areal heating elements of an element row.

41. The device according to claim 39, wherein at least one of:
said row spacing is defined by a distance between two adjacent areal heating elements of adjacent element rows and is 1 mm to 5 mm, or
said intermediate distance between two adjacent areal heating elements of an element row is 0.5 mm to 5 mm.

42. The device according to claim 40, wherein at least one of:
said row spacing is defined by a distance between two adjacent areal heating elements of adjacent element rows and is 1 mm to 5 mm, or
said intermediate distance between two adjacent areal heating elements of an element row is 0.5 mm to 5 mm.

43. The device according to claim 32, wherein said first main line and said second main line of said film layer conductor grid are each laterally and substantially perpendicularly aligned with said element rows and have a main line width of from 1 mm to 5 mm.

44. The device according to claim 43, wherein said main line width is from 1 mm to 3 mm.

45. The device according to claim 32, wherein all of said stub lines and said other stub lines are aligned substantially parallel and are associated with said element rows.

46. The device according to claim 45, wherein each of said stub lines and said other stub lines have a stub width of 0.3 mm to 1 mm.

47. The device according to claim 32, wherein one of said stub lines and one of said other stub lines of an element row have a stub line spacing of at least 1 mm, and said stub line spacing between said one of said stub lines and said one of said other stub lines is always selected to cause said one of said stub lines and said one of said other stub lines to run within an area of an associated areal heating element.

48. The device according to claim 30, wherein said areal heating elements formed by at least one of said film layer resistors or said film layer conductor grid have a layer thickness of 0.5 μm to 30 μm.

49. The device according to claim 30, wherein said areal heating elements formed by at least one of said film layer resistors or said film layer conductor grid have a layer thickness of 5 μm to 25 μm.

50. The device according to claim 30, wherein said areal heating elements formed by at least one of said film layer resistors or said film layer conductor grid have a layer thickness of 10 μm to 20 μm.

51. The device according to claim 32, which further comprises:
a container holder having said electrical connection element;
said base carrier layer being formed by said container or being a component of said container;
said container being detachably connected to said container holder; and
said container being electrically connectable to said container holder.

52. The device according to claim 51, which further comprises an electrical contact device or an electrical contact device with plug-in elements, disposed on said first and second main lines, said first and second main lines being directly or indirectly connectable to said electrical connection element by said electrical contact device for electrical power supply of said areal heating elements.

53. The device according to claim 51, wherein:
said container holder is formed as a plug component; and
said electrical connection element has projecting plug contacts being pluggable into an electrical socket for supplying electrical power to said areal heating elements and for holding said plug component and said container connected to said plug component.

54. The device according to claim 51, which further comprises:
an areal heat transfer element;
said base carrier layer directly forming an outer side of a container wall part or a non-conductive intermediate layer connected to a container wall part; and
a heat transfer region having a capillary element region configured to be heated and adjoining said base carrier layer or adjoining said base carrier layer in an areal manner with said areal heating elements or with an interposition of said areal heat transfer element.

55. The device according to claim 54, which further comprises a capillary element being a U-shaped wick in contact with the substance to be dispensed, said U-shaped wick having two U-legs disposed in said container and a U-base part forming said at least one substance dispensing area.

56. The device according to claim 30, which further comprises:
a container holder, said container being detachably connected to said container holder;
said container holder being formed by a housing component having an interior, said container being at least partially accommodated in said housing component;
said base carrier layer with said areal heating elements being part of said housing component and disposed in said interior of said housing component in a region of said inserted container; and
said housing component having said electrical connection element, said container being electrically connected to said electrical connection element.

57. The device according to claim 56, wherein said base carrier layer with said areal heating elements is disposed in a region of a substance dispensing area of a capillary element to be heated, or is connected to a free wick end of a wick forming a capillary element, said free wick end protruding from said container.

58. The device according to claim 56, wherein:
said housing component is configured as a connector component;
said container holder is formed as a plug component; and
said electrical connection element has projecting plug contacts being pluggable into an electrical socket for supplying electrical power to said areal heating elements and for holding said plug component and said container connected to said plug component.

59. A method of making a device for dispensing volatile substances, the method comprising:
providing a container for the substance to be dispensed;
providing at least one substance dispensing area;
providing at least one capillary element being disposed at least partially in the container, being in contact with the substance to be dispensed and conveying the substance to be dispensed by capillary action to the at least one substance dispensing area;
providing an electrical heating device having at least one electrical heating resistor disposed in a region of the at least one capillary element; and
providing an electrical connection element for supplying the electrical heating device with electrical energy;
producing the heating resistor with at least two element rows spaced apart from one another, each element row having at least one areal heating element;
applying the areal heating elements as film layer resistors to an electrically non-conductive base carrier layer; and
applying an electrically conductive film layer conductor grid to the base carrier layer for supplying electrical energy to the areal heating elements.

60. The method according to claim 59, which further comprises providing the at least one areal heating element as at least one PTC areal heating element.

61. The method according to claim 59, which further comprises:
placing a first main line and a second main line of the film layer conductor grid on opposite sides of the at least two rows of elements for connecting each of the first and second main lines to a respective pole having a different voltage of the electrical connection element;
routing a respective stub line starting from the first main line and running along each row of elements while electrically contacting the at least one areal heating element of each row of elements;
routing a respective other stub line starting from the second opposite main line and running along each row of elements while electrically contacting the at least one areal heating element of each row of elements; and
providing each of the stub lines starting from the first main line and the other stub lines starting from the second main line with a stub line spacing between associated areal heating elements of an element row.

62. The method according to claim 59, which further comprises forming the areal heating elements forming film layer resistances from an electrically conductive ink or paint having a resistance function, or a PTC resistance function, and applying to the base carrier layer as a film layer or printing and drying.

63. The method according to claim 59, which further comprises forming the electrically conductive film layer conductor grid from an electrically conductive ink or ink having a current-carrying function or ink including silver, being applied to the base carrier layer as a film layer or printed on and dried.

64. The method according to claim 61, which further comprises:
applying the areal heating elements in front of the film layer conductor grid in order to lie below the stub lines, or applying the film layer conductor grid before the areal heating elements resulting in the areal heating elements being applied to the stub lines lying underneath.

65. The method according to claim 59, which further comprises manufacturing the areal heating elements identically with a rectangular or square shape.

66. The method according to claim 59, which further comprises at least one of:
applying at least two areal heating elements spaced apart from one another by intermediate distances in an element row, or
providing an equal number of areal heating elements in each element row.

67. The method according to claim 59, which further comprises aligning two to six element rows each having two to five areal heating elements in parallel and with at least one of:
an equal row spacing between opposite areal heating elements of adjacent element rows, or
an identical intermediate spacing between adjacent areal heating elements of an element row.

68. The method according to claim 59, which further comprises aligning two or four element rows each having three or four areal heating elements in parallel and with at least one of:
an equal row spacing between opposite areal heating elements of adjacent element rows, or
an identical intermediate spacing between adjacent areal heating elements of an element row.
